(19) United States
(12) Patent Application Publication
(10) Pub. No.: US 2017/0281703 A1
(43) Pub. Date: Oct. 5, 2017

(54) NUTRITIONAL SUPPLEMENT AND RELATED METHOD FOR ACTIVATING A SUBJECT'S ANTIOXIDANT SYSTEM

(71) Applicant: Access Business Group International LLC, Ada, MI (US)

(72) Inventors: Kelly M. Glynn, Grand Rapids, MI (US); John F. Rebhun, Greenville, MI (US); Samantha Roloff, Grand Rapids, MI (US); Yingqin Li, Murrieta, CA (US); Dawna Venzon, Brea, CA (US)

(21) Appl. No.: 15/464,695

(22) Filed: Mar. 21, 2017

Related U.S. Application Data
(60) Provisional application No. 62/314,775, filed on Mar. 29, 2016.

(51) Int. Cl. A61K 36/484; A61K 31/12; A61K 31/353

(52) U.S. Cl. CPC A61K 36/484; A61K 31/353; A61K 31/12

(57) ABSTRACT

A nutritional supplement comprises components (A), (B) and (C), and optionally, additional component(s) (D). The nutritional supplement can activate a subject's antioxidant system, more specifically the subject's antioxidant response element (ARE). In one embodiment, the nutritional supplement comprises: (A) glabridin; (B) licochalcone A; and (C) licorice root flavonoids. In another, the nutritional supplement comprises: (A) a first licorice root extract comprising glabridin; (B) a second licorice root extract comprising licochalcone A; and (C) a third licorice root extract comprising flavonoids. Components (A), (B) and (C) are in a combined amount sufficient to activate the subject's antioxidant system after ingestion. In addition, components (A), (B) and (C) are present in a weight ratio (A:B:C) such that their combined effect is greater than the sum of their separate effects regarding activation of the subject's antioxidant system after ingestion of the nutritional supplement.

### FIELD OF THE INVENTION

The present invention generally relates to a nutritional supplement suitable for ingestion, and more specifically to a nutritional supplement comprising synergistic amounts of different licorice root extracts and/or different compounds/constituents derived from licorice roots and to a method of utilizing the nutritional supplement for activating a subject's antioxidant system (e.g. the subject's antioxidant response element (ARE)).

### BACKGROUND OF THE INVENTION

Licorice root has long been used in medicinal and edible applications and has been studied extensively for its biological activity. Many of the chemical constituencies within licorice root have been identified (e.g. glycyrrhizin), and their mechanisms of action have been explored in anti-inflammatory, antimicrobial and antiviral, anti-oxidative, cardiovascular, anticancer and skin-related activities.

Unfortunately, there has been minimal investigation of how certain constituents of licorice root may or may not impact anti-oxidative activities in the human body. In addition, there has been minimal investigation of how certain constituents of licorice root may interact with one another when combined in manners and/or amounts not found naturally.

In view of the foregoing, there remains an opportunity to provide improved nutritional supplements based on licorice root extracts and/or different compounds/constituents derived from licorice roots. There also remains an opportunity to provide methods utilizing such nutritional supplements.

### SUMMARY OF THE INVENTION

A nutritional supplement is provided. The nutritional supplement comprises components (A), (B) and (C). In one embodiment, the nutritional supplement comprises: (A) glabridin; (B) licochalcone A; and (C) licorice root flavonoids.

In another embodiment, the nutritional supplement comprises: (A) a first licorice root extract comprising glabridin; (B) a second licorice root extract different from the first licorice root extract; and (C) a third licorice root extract different from the first licorice root extract and different from the second licorice root extract. For example, the first licorice root extract comprises glabridin. The second licorice root extract comprises licochalcone A. The third licorice root extract comprises flavonoids.

Components (A), (B) and (C) are present in a combined amount sufficient to activate a subject's antioxidant system after ingestion of the nutritional supplement. In addition, components (A), (B) and (C) are present in a weight ratio (A:B:C) such that their combined effect is greater than the sum of their separate effects with respect to activation of the subject's antioxidant system after ingestion of the nutritional supplement. Said another way, certain A:B:C weight ratios provide a synergistic effect with respect to activation of the subject's antioxidant system after ingestion of the nutritional supplement.

These and other objects, advantages, and features of the present invention will be more fully understood and appreciated by reference to the description of the current embodiments and drawings.

Before the embodiments of the present invention are explained in detail, it is to be understood that the present invention is not limited to the details of operation or to the details of construction and the arrangement of the components set forth in the following description or illustrated in the drawings. The present invention may be implemented in various other embodiments and be capable of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the present invention to any specific order or number of components. Nor should the use of enumeration be construed as excluding from the scope of the present invention any additional components or steps that might be combined with or into the enumerated components or steps.

### BRIEF DESCRIPTION OF THE DRAWINGS

FIG. 1A is a high-performance liquid chromatography (HPLC) chart illustrating the chromatogram of (A) a first licorice root extract comprising glabridin;

FIG. 1B is another HPLC chart illustrating a chromatogram of the first licorice root extract where a different HPLC method was utilized;

FIG. 2A is a HPLC chart illustrating the chromatogram of (B) a second licorice root extract comprising licochalcone A;

FIG. 2B is another HPLC chart illustrating a chromatogram of the second licorice root extract where a different HPLC method was utilized;

FIG. 3A is a HPLC chart illustrating the chromatogram of (C) a third licorice root extract comprising flavonoids;

FIG. 3B is another HPLC chart illustrating a chromatogram of the third licorice root extract where a different HPLC method was utilized;

FIG. 4 is a dose response curve of the third licorice root extract of FIG. 3;

FIG. 5 is a dose response curve of the first licorice root extract of FIG. 1;

FIG. 6 is a dose response curve of the second licorice root extract of FIG. 2;

FIG. 7 is a bar chart illustrating increase in antioxidant response element (ARE) for certain amounts of the individual licorice root extracts of FIGS. 1 to 3, an expected additive effect when the licorice root extracts are combined in a particular weight ratio, and an observed synergistic effect when the licorice root extracts are actually combined in the particular weight ratio;

FIG. 8 is another bar chart illustrating increase in ARE for certain amounts of the individual licorice root extracts of FIGS. 1 to 3, an expected additive effect when the licorice root extracts are combined in another particular weight ratio, and an observed synergistic effect when the licorice root extracts are actually combined in the particular weight ratio;

FIG. 9 is another bar chart illustrating increase in ARE for certain amounts of the individual licorice root extracts of FIGS. 1 to 3, an expected additive effect when the licorice root extracts are combined in another particular weight ratio, and an observed synergistic effect when the licorice root extracts are actually combined in the particular weight ratio;

FIG. 10 is another bar chart illustrating increase in ARE for certain amounts of the individual licorice root extracts of FIGS. 1 to 3, an expected additive effect when the licorice root extracts are combined in another particular weight ratio, and an observed synergistic effect when the licorice root extracts are actually combined in the particular weight ratio;

FIG. 11 is another bar chart illustrating increase in ARE for certain amounts of the individual licorice root extracts of FIGS. 1 to 3, an expected additive effect when the licorice root extracts are combined in another particular weight ratio, and an observed synergistic effect when the licorice root extracts are actually combined in the particular weight ratio;

FIG. 12 is another bar chart illustrating increase in ARE for certain amounts of the individual licorice root extracts of FIGS. 1 to 3, an expected additive effect when the licorice root extracts are combined in another particular weight ratio, and an observed synergistic effect when the licorice root extracts are actually combined in the particular weight ratio;

FIG. 13 is another bar chart illustrating increase in ARE for certain amounts of the individual licorice root extracts of FIGS. 1 to 3, an expected additive effect when the licorice root extracts are combined in a comparative weight ratio, and lack of an observed synergistic effect when the licorice root extracts are actually combined in the comparative weight ratio; and

FIG. 14 is another bar chart illustrating increase in ARE for certain amounts of the individual licorice root extracts of FIGS. 1 to 3, an expected additive effect when the licorice root extracts are combined in a comparative weight ratio, and lack of an observed synergistic effect when the licorice root extracts are actually combined in the comparative weight ratio.

### DETAILED DESCRIPTION OF THE CURRENT EMBODIMENTS

The current embodiments provide a nutritional supplement as disclosed herein. After ingestion, the nutritional supplement is useful for activating a subject's antioxidant system. Specifically, the subject's antioxidant response element (ARE) activates after ingestion of the nutritional supplement. Such activation may be near instantaneous or delayed. In some cases, the activation can be delayed for a period of time required for the nutritional supplement to be at least partially digested and/or metabolized after ingestion.

Without being bound or limited to any particular theory, it is thought that activation (e.g. transcriptional activation) of the ARE plays an important role in modulating oxidative stress and providing cytoprotection against pro-oxidant stimuli. Specifically, it is thought that ARE is the primary activator of the mammalian antioxidant system. ARE is a conserved DNA sequence to which the transcription factor, Nrf2, binds and activates antioxidant gene expression in mammalian cells. Nuclear factor (erythroid-derived 2)-like 2, also known as NFE2L2 or Nrf2, is a transcription factor that in humans is encoded by the NFE2L2 gene. Nrf2 is a basic leucine zipper (bZIP) protein that regulates the expression of antioxidant proteins that protect against oxidative damage triggered by injury and inflammation.

The nutritional supplement may also be referred to as a “phytonutrient supplement,” “phytonutrient composition” or “dietary supplement,” or simply as a “supplement” or “composition.” It is to be appreciated that the nutritional supplement is not limited to a particular use, such as use only for nutrition, dietary, and/or supplemental needs. In addition, the nutritional supplement may also provide benefits in addition to activation of the ARE. For example, while studying the nutritional supplement and components thereof, in-vitro activity was observed in peroxisome proliferator-activated receptor gamma (PPARg) and glycation inhibition assays by the inventors.

The nutritional supplement generally comprises three primary components, and optionally, one or more secondary components. Specifically, the nutritional supplement comprises component (A), component (B) and component (C), each as described herein. Optionally, the nutritional supplement may further comprise one or more additional components, e.g. component (D), such as one or more conventional additives understood in the art. The primary components are described further below, along with optional secondary components of the nutritional supplement and additional aspects thereof. As used herein, it is to be appreciated that the designations “(A),” “(B)” and “(C)” or “first,” “second” and “third” are not to be construed as requiring a particular order or indicating a particular importance of one component relative to the other.

In various embodiments, the nutritional supplement consists essentially of components (A), (B) and (C). As used herein, the phrase “consisting essentially of” generally encompasses the specifically recited elements/components for a particular embodiment. Further, the phrase “consisting essentially of” generally encompasses and allows for the presence of additional or optional elements/components that do not materially impact the basic and/or novel characteristics of that particular embodiment. In certain embodiments, “consisting essentially of” allows for the presence of ≦10, ≦5, or ≦1, weight percent (wt %) of additional or optional components based on the total weight of the nutritional supplement. In other embodiments, the nutritional supplement consists of components (A), (B) and (C).

In various embodiments, each of components (A), (B) and (C) are individually derived from licorice plants, more typically from roots of licorice plants. These embodiments are detailed further below. In general, licorice plants are herbaceous perennial legumes native to southern Europe, India, and parts of Asia. Glycyrrhiza is a genus of about 18 accepted species in the legume family (Fabaceae). Although there are more species found throughout the world, notable licorice species include: Glycyrrhiza (G.) acanthocarpa; G. aspera; G. astragalina; G. bucharica; G. echinata; G. eglandulosa; G. foetida; G. foetidissima; G. glabra; G. gontscharovii; G. iconica; G. inflata; G. korshinskyi; G. lepidota; G. pallidiflora; G. squamulosa; G. triphylla; G. uralensis; and G. yunnanensis.

At least one of the aforementioned species (e.g. G. glabra), more typically at least two or three of the aforementioned species (e.g. G. glabra and G. inflata), are useful for providing components (A), (B) and (C). In various embodiments, each of components (A), (B) and (C) are provided by/derived from different licorice root extracts. Components (A), (B) and (C) may simply be referred to herein as the “licorice root extracts.” The licorice root extracts may be “different” based on their source licorice species, particular method of extraction, or combinations thereof. For example, differing species or methods of extraction can provide extracts with different constituent profiles, i.e., differing makeups and amounts of chemical compounds. In various embodiments, components (A), (B) and (C) are different based on a combination of different source licorice species and further by different methods of extraction.

Suitable licorice root extracts can be obtained via conventional extraction methods understood in the art, such as by water (e.g. steam) extraction or by solvent (e.g. alcohol) extraction. The nutritional supplement is not limited to a particular extraction method; however, alcohol (e.g. ethanol) extraction is generally utilized in various embodiments. Exemplarily extraction methods are described below.

There are a variety of extraction methods that may be used to produce licorice root extracts suitable for the nutritional supplement. These methods include, but are not limited to, the extraction methods disclosed in U.S. Pat. No. 7,897,184 to Rana et al., which is hereby incorporated by reference in its entirety. While extraction solvents described specifically mention ethanol, it should be understood that other alcohols such as, but not limited to, isopropyl alcohol, ethyl alcohol, and/or methyl alcohol may be used in addition to or as an alternative to ethanol. Exemplary alcoholic solvents include, but are not limited to, C₁ to C₄ alcohols, such as methanol, ethanol, propanol, isopropanol, and butanol; hydro-alcohols or mixtures of alcohol and water, including hydro-ethanol; polyhydric alcohols such as propylene glycol and butylene glycol; and fatty alcohols. Any of these alcoholic solvents may be used. Other solvents such as, but not limited to, acetone may also be used as an extraction solvent. Solvent-water blends, e.g. alcohol-water and/or acetone-water blends, of any ratio, may also be used. In various embodiments, the solvent is one in which the resulting extract and/or a subsequent form thereof (e.g. extract powder) is suitable for ingestion. For example, the solvent is water or ethanol.

In one example, the licorice root extracts can be obtained using an organic solvent extraction technique. In another example, solvent sequential fractionation can be used to obtain the licorice root extracts. Total hydro-ethanolic extraction techniques can also be used to obtain the licorice root extracts. Generally, this is referred to as a lump-sum extraction. The extract generated in the process will contain a broad variety of phytochemicals present in the extracted material including fat and water soluble phytochemicals. Following collection of the extract solution, the solvent will be evaporated, resulting in the extract.

Total ethanol extraction may also be used. This technique uses ethanol as the solvent. This extraction technique generates an extract that may include fat soluble and/or lipophilic compounds in addition to water soluble compounds. Total methanol extraction may also be used in a similar manner with similar results. In various embodiments, each of the licorice root extracts is individually obtained by alcohol extraction of plant material, e.g. roots, of the species G. glabra and/or G. inflata, more typically G. glabra and G. inflata.

Another example of an extraction technique that can be used to obtain the licorice root extracts is supercritical fluid carbon dioxide extraction (SFE). In this extraction procedure, the material to be extracted is not exposed to any organic solvents. Rather, the extraction solvent is carbon dioxide (CO₂), with or without a modifier, in super-critical conditions (e.g. >31.3° C. and >73.8 bar). Those of skill in the art will appreciate that temperature and pressure conditions can be varied to obtain the best yield of extract. This technique generates an extract of fat soluble and/or lipophilic compounds, similar to total hexane and ethyl acetate extraction techniques, which may also be used.

Each of the extraction methods above also may include and/or be utilized in combination with one or more additional processing steps understood in the art. For example, plant material may be comminuted, smashed, ground, etc. There also may be one or more filtration steps to remove, for example, cellulosic/fibrous or other solid materials. There also may be one or more purification steps to remove, for example, certain constituents and/or contaminants. Such purification may be accomplished, for example, by distillation, evaporation, centrifugation, etc. There also may be one or more concentration and/or drying steps to remove water and/or other volatiles, e.g. alcohol, lighter compounds, VOCs, etc. Moreover, acids and/or bases may be added to adjust pH or neutralize. Depending on the desired form of the final/end extract, one can also utilize various additional steps understood in the art, such as screening, pressing, milling, grinding, mixing, dispersing, etc. It is to be appreciated that combinations of these additional processing steps in duplicative and/or different orders is also contemplated.

In various embodiments, components (A), (B) and (C) are each individually derived via extraction of root material obtained from different licorice plants, alternatively via ethanolic extraction of dry root material obtained from different licorice plants. Licorice root materials that can be utilized for extraction include root sticks, root slivers, root chips, and/or root powder, as well as such materials with and/or without bark. Suitable licorice root extracts are commercially available from a number of suppliers, including from Beijing Gingko Group (BGG) North America, Inc. of Irvine, Calif.

In various embodiments, component (B) is obtained via a first extraction method and component (C) is obtained via a second extraction method. The second extraction method is different from the first extraction method in at least one way. In certain embodiments, the first extraction method includes an additional step of purifying the licorice extract relative to the second extraction method. Such purification can be accomplished, for example, via a column. Component (A) may be obtained via the first or second extraction methods, similar methods, or an alternate method altogether. The first and second extraction methods are useful in instances where the same source species of licorice plant is utilized, but where a different makeup of constituents and/or majority constituent is desired. For example, certain constituents can be removed or concentrated utilizing purification techniques understood in the art.

In various embodiments, component (A) comprises glabridin, alternatively consists essentially of glabridin. While component (A) may consist of glabridin outright, other secondary components can generally be utilized in the nutritional supplement to provide or improve, for example, aesthetics and/or ingestability of the nutritional supplement.

Typically, component (A) is derived from and/or is a first licorice root extract comprising glabridin. In certain embodiments, the first licorice root extract is derived from the species Glycyrrhiza glabra L. The first licorice root extract may contain additional constituents, such as, but not limited to, at least one of licochalcone A, glycyrrhizin and liquiritin, alternatively component (A) comprises glabridin, licochalcone A, glycyrrhizin, and liquiritin. In these embodiments, glabridin is the majority constituent of the first licorice root extract/component (A).

In certain embodiments, component (A) comprises the following constituents: about 4 to about 6, alternatively about 4 to about 5, alternatively about 4, wt % glabridin; about 0 to about 1, alternatively about 0.01 to about 0.1, wt % licochalcone A; and about 15 to about 25, alternatively about 20, wt % total flavonoids.

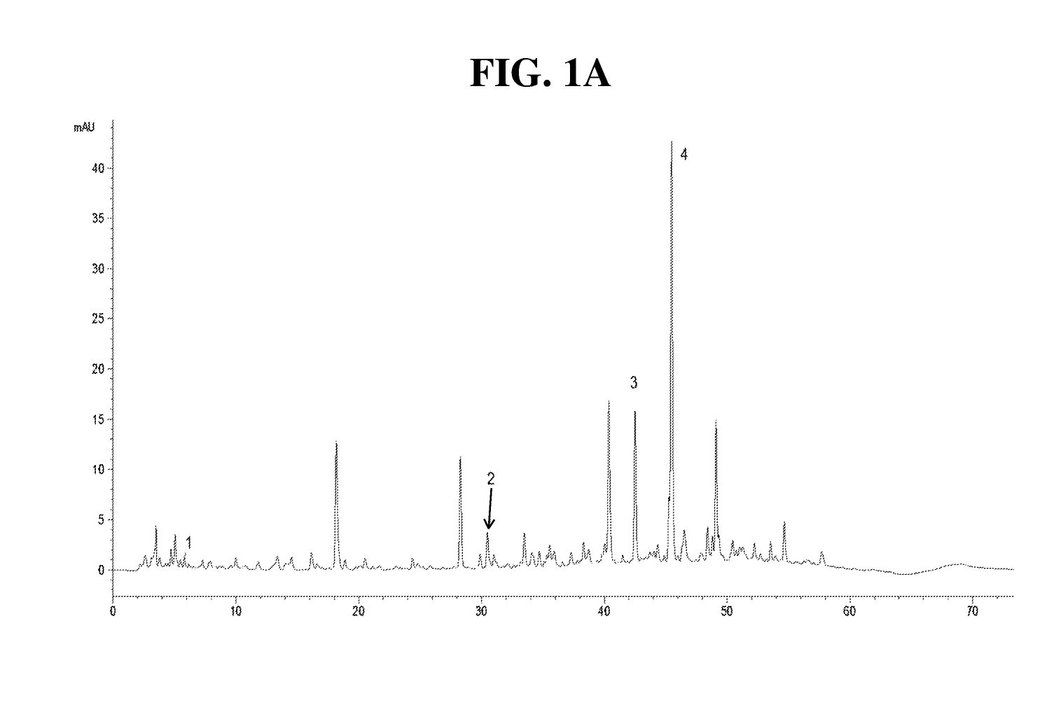

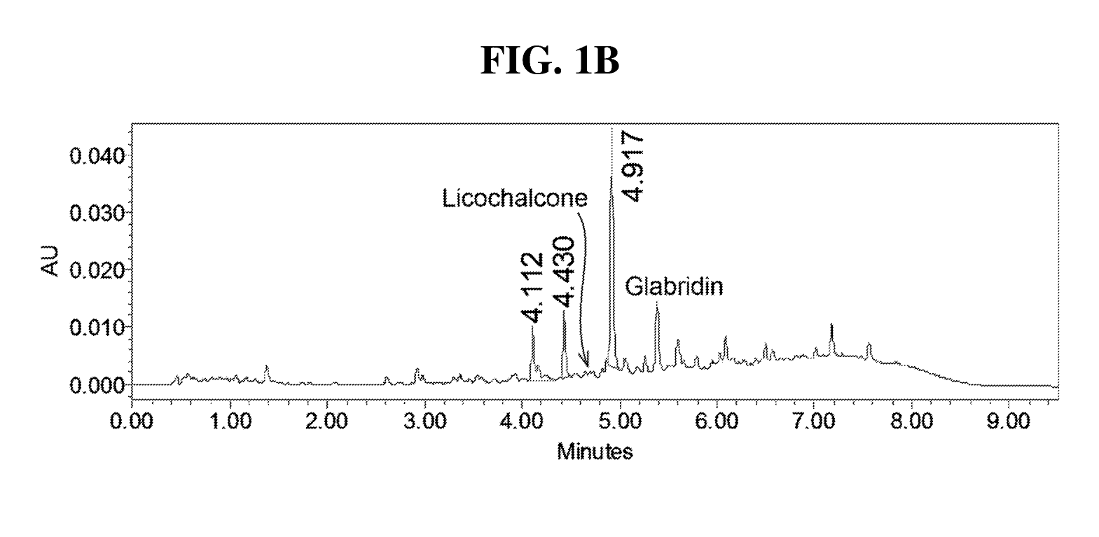

In certain embodiments, the first licorice root extract is obtained from BGG and generally has chromatograms as illustrated in FIG. 1. Referring to FIG. 1A, the first licorice root extract has four enumerated peaks, with numeral 1 designating liquiritin, numeral 2 designating glycyrrhizin, numeral 3 designating licochalcone A, and numeral 4 designating glabridin. Glabridin is an isoflavane, a type of isoflavonoid. Licochalcone A is a chalconoid, a type of natural phenol. Glycyrrhizin is also known as glycyrrhizic acid or glycyrrhizinic acid, and structurally, glycyrrhizin is a saponin. Liquiritin is the 4′-O-glucoside of the flavanone liquiritigenin. Referring to FIG. 1B, which is generated utilizing an alternate HPLC method, the first licorice root extract has three enumerated peaks, a peak designating licochalcone A, and a peak designating glabridin.

In various embodiments, component (A) is present in an amount of from about 5 to about 45 parts by weight, alternatively about 8 to about 42 parts by weight, based on 100 parts by weight of the nutritional supplement. In certain embodiments, component (A) is present in an amount of from: about 30 to about 35, alternatively about 33; about 10 to about 20, alternatively about 14 to about 16, alternatively about 14, alternatively about 16; about 35 to about 45, alternatively about 42; about 5 to about 15, alternatively about 8; parts by weight, based on 100 parts by weight of the nutritional supplement. Various subranges and amounts of component (A) between about 5 and about 45 parts by weight of the nutritional supplement, as well as amounts that are less than or greater than these amounts, are also contemplated. Optionally, the amounts above can be adjusted or normalized to account the inclusion of optional secondary components.

In various embodiments, component (B) comprises licochalcone A, alternatively consists essentially of licochalcone A. While component (B) may consist of licochalcone A outright, other secondary components can generally be utilized in the nutritional supplement as a buffer to improve, for example, aesthetics and/or ingestability of the nutritional supplement.

Typically, component (B) is derived from and/or is a second licorice root extract comprising licochalcone A. The second licorice root extract is different from the first licorice root extract. In certain embodiments, the second licorice root extract is derived from the species Glycyrrhiza inflata B. The second licorice root extract may contain additional constituents, such as, but not limited to, at least one of glabridin, glycyrrhizin and liquiritin, alternatively component (B) comprises licochalcone A, glabridin, glycyrrhizin, and liquiritin. In these embodiments, licochalcone A is the majority constituent of the second licorice root extract/component (B).

In certain embodiments, component (B) comprises the following constituents: about 0 to about 1, alternatively about 0.01 to about 0.5, wt % glabridin; about 15 to about 25, alternatively about 20 to about 23, alternatively about 21, wt % licochalcone A; and about 85 to about 95, alternatively about 91 to about 93, wt % total flavonoids.

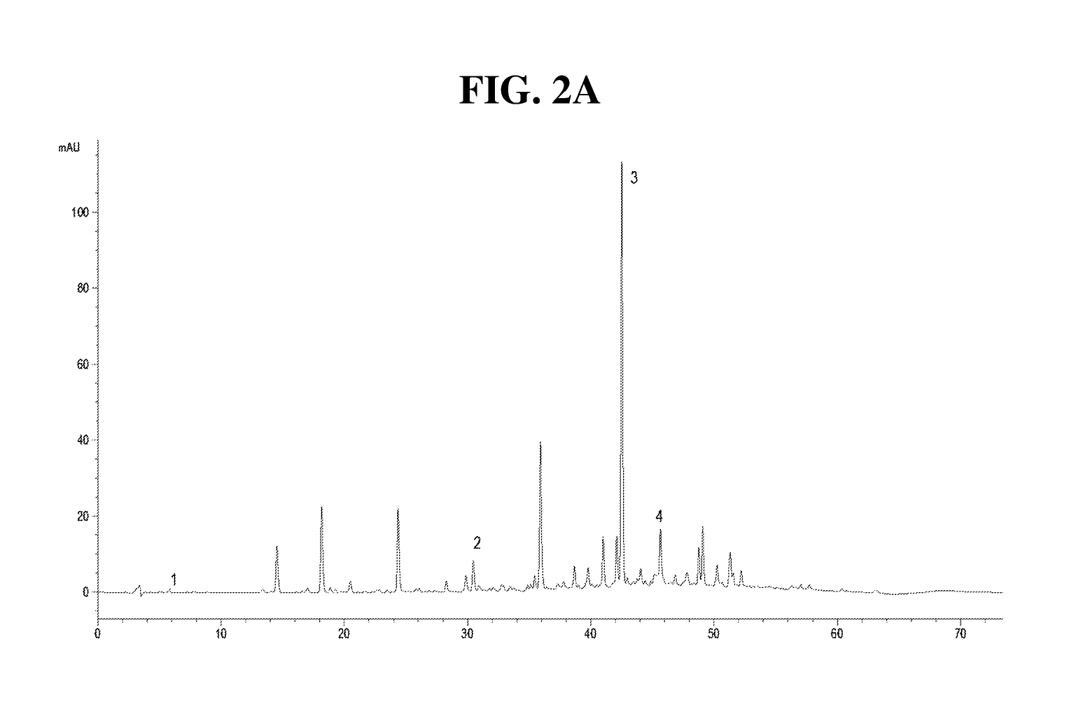

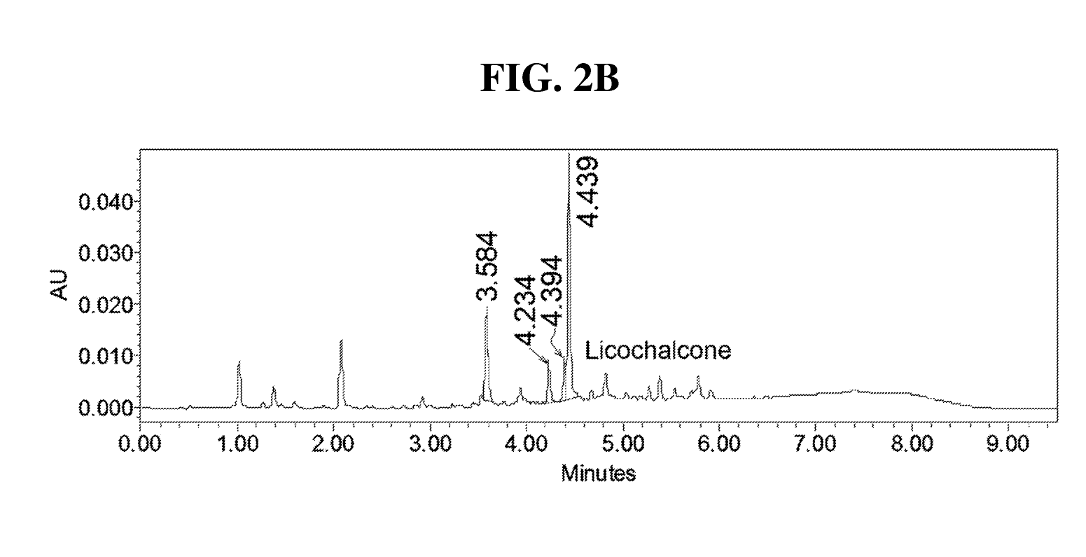

In certain embodiments, the second licorice root extract is obtained from BGG and generally has chromatograms as illustrated in FIG. 2. Referring to FIG. 2A, the second licorice root extract has four enumerated peaks, with numeral 1 designating liquiritin, numeral 2 designating glycyrrhizin, numeral 3 designating licochalcone A, and numeral 4 designating glabridin. Referring to FIG. 2B, which is generated utilizing an alternate HPLC method, the second licorice root extract has four enumerated peaks, and a peak designating licochalcone A.

In various embodiments, component (B) is present in an amount of from about 5 to about 45 parts by weight, alternatively about 8 to about 42 parts by weight, based on 100 parts by weight of the nutritional supplement. In certain embodiments, component (B) is present in an amount of from: about 30 to about 35, alternatively about 33; about 10 to about 20, alternatively about 14 to about 16, alternatively about 14, alternatively about 16; about 35 to about 45, alternatively about 42; about 5 to about 15, alternatively about 8; parts by weight, based on 100 parts by weight of the nutritional supplement. Various subranges and amounts of component (B) between about 5 and about 45 parts by weight of the nutritional supplement, as well as amounts that are less than or greater than these amounts, are also contemplated. Optionally, the amounts above can be adjusted or normalized to account the inclusion of optional secondary components.

In various embodiments, component (C) comprises at least one of glabridin, licochalcone A, glycyrrhizin and liquiritin, alternatively component (C) comprises glabridin, licochalcone A, glycyrrhizin, and liquiritin. In further embodiments, component (C) consists essentially of at least one of glabridin, licochalcone A, glycyrrhizin and liquiritin, alternatively component (C) consists essentially of glabridin, licochalcone A, glycyrrhizin, and liquiritin. While component (C) may consist of at least one of glabridin, licochalcone A, glycyrrhizin and liquiritin outright, other secondary components can generally be utilized in the nutritional supplement as a buffer to improve, for example, aesthetics and/or ingestability of the nutritional supplement.

Typically, component (C) is derived from and/or component (C) is a third licorice root extract comprising licorice flavonoids. The third licorice root extract is different from the first licorice root extract. The third licorice root extract is also different from the second licorice root extract. In certain embodiments, the third licorice root extract is derived from the species Glycyrrhiza inflata B. In these embodiments, licochalcone A is typically the majority constituent of the third licorice root extract/component (C), optionally followed next by glabridin, then by glycyrrhizin, then by liquiritin.

In certain embodiments, component (C) comprises the following constituents: about 0 to about 1, alternatively about 0.01 to about 0.1, wt % glabridin; about 5 to about 15, alternatively about 10 to about 12, wt % licochalcone A; and about 65 to about 85, alternatively about 70 to about 83, wt % total flavonoids.

In embodiments where both the second and third licorice root extracts are derived from the same species, e.g. Glycyrrhiza inflate B., the extracts are differentiated by at least one of different source plants and/or different extraction techniques. For example, components (B) and (C) may be obtained via the first and second extraction methods introduced above.

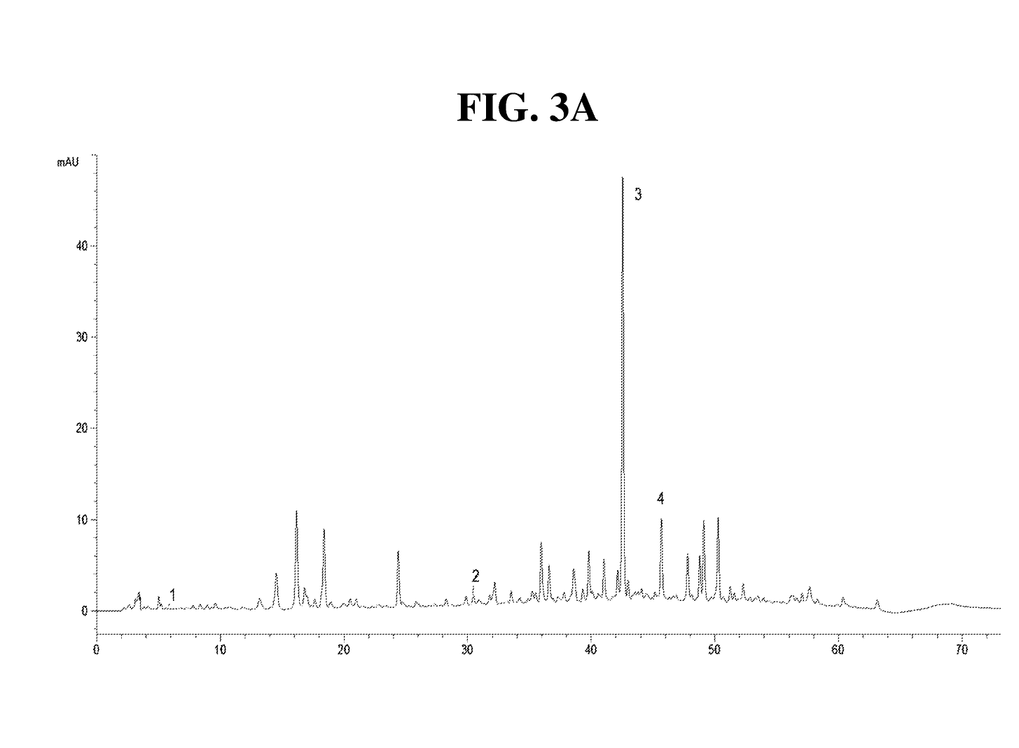

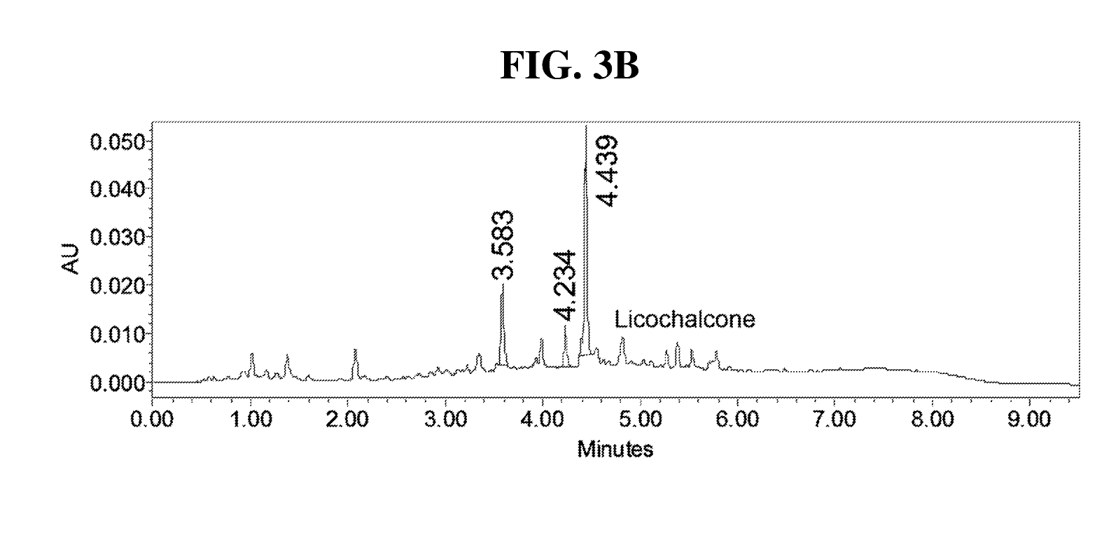

In certain embodiments, the third licorice root extract is obtained from BGG and generally has chromatograms as illustrated in FIG. 3. Referring to FIG. 3A, the third licorice root extract has four enumerated peaks, with numeral 1 designating liquiritin, numeral 2 designating glycyrrhizin, numeral 3 designating licochalcone A, and numeral 4 designating glabridin. Referring to FIG. 3B, which is generated utilizing an alternate HPLC method, the third licorice root extract has three enumerated peaks, and a peak designating licochalcone A.

In various embodiments, component (C) is present in an amount of from about 10 to about 90 parts by weight, alternatively about 16 to about 83 parts by weight, based on 100 parts by weight of the nutritional supplement. In certain embodiments, component (C) is present in an amount of from: about 30 to about 35, alternatively about 33; about 10 to about 20, alternatively about 14 to about 16, alternatively about 14, alternatively about 16; about 35 to about 45, alternatively about 42; about 5 to about 15, alternatively about 8; about 65 to about 75, alternatively about 71; about 80 to about 85, alternatively about 83; parts by weight, based on 100 parts by weight of the nutritional supplement. Various subranges and amounts of component (C) between about 10 and about 90 parts by weight of the nutritional supplement, as well as amounts that are less than or greater than these amounts, are also contemplated. Optionally, the amounts above can be adjusted or normalized to account the inclusion of optional secondary components.

In various embodiments, component (A) is present in an amount less than a combined amount of components (B) and (C). Said another way, component (A) is typically present in an amount <50 wt %, alternatively <45 wt %, alternatively <40 wt %, alternatively <35 wt %, alternatively <30 wt %, based on 100 parts by weight of the nutritional supplement.

In certain embodiments, components (A), (B) and (C) are present in a weight ratio (A:B:C) of about 1:3:3, alternately about 1:2.6:2.6. In other embodiments, components (A), (B) and (C) are present in a weight ratio (A:B:C) of about 3:3:1, alternatively about 2.6:2.6:1. In yet other embodiments, components (A), (B) and (C) are present in a weight ratio (A:B:C) of about 1:1:10, alternatively about 1:1:10.4. In yet other embodiments, the components (A), (B) and (C) are present in a weight ratio (A:B:C) of about 3:1:3, alternatively about 2.6:1:2.6. In yet other embodiments, components (A), (B) and (C) are present in a weight ratio (A:B:C) of about 1:1:5. In a further embodiment, components (A), (B) and (C) are present in a weight ratio (A:B:C) of about 1:1:1. Various ranges and subranges of (A:B:C) weight ratios in view of the foregoing explicit ratios are also contemplated.

As introduced above, the nutritional supplement is useful for antioxidant purposes. In various embodiments, components (A), (B) and (C) are present in a combined amount sufficient to activate the subject's antioxidant system after ingestion of the nutritional supplement. In addition, components (A), (B) and (C) are present in a weight ratio (A:B:C) such that their combined effect is greater than the sum of their separate effects with respect to activation of the subject's antioxidant system after ingestion of the nutritional supplement. Quite surprisingly and unexpectedly, the inventors discovered a synergy between components (A), (B) and (C) in this regard, especially with respect to substantially improved activation of ARE. Specific improvements can be better appreciated with reference to the EXAMPLES section further below.

Optionally, the nutritional supplement may include (D) one or more secondary components, such as one or more additives. Suitable additives include those understood in the art. Such additives may be utilized alone or in combination. Various optional additives are described in greater detail below. If utilized, the additive(s) may be present in the nutritional supplement in various amounts.

In various embodiments, the nutritional supplement comprises at least one of vitamins, minerals, and specialty ingredients known to improve the body's natural defenses against oxidants or free radicals. Suitable vitamins include, but are not limited to, vitamin A, vitamin C, vitamin D, vitamin E, vitamin K, vitamin B6, vitamin B12, thiamin, riboflavin, niacin, folic acid, biotin, pantothenic acid, and combinations thereof. Suitable minerals include, but are not limited to, calcium, magnesium, iodine, potassium, copper, zinc, phosphorus, manganese, chromium, selenium, molybdenum, vanadium, boron, and combinations thereof. Other vitamins and minerals may also be used. Additional ingredients which may be included in the nutritional supplement are, for example, methylsulfonylmethane (MSM) and/or a-lipoic acid.

In certain embodiments, the nutritional supplement may include one or more other extracts different from each of components (A), (B) and (C). Suitable extracts include, but are not limited to, extracts of grape seed (e.g. from the genus Vitis), goji berry (e.g. from the genus Lycium barbarum and/or Lycium chinense), rose hip (e.g. from the genus Rosa), and combinations thereof. Additional extracts suitable for the nutritional supplement include extracts of aronia berry (or “chokeberries,” e.g. from the species Aronia melanocarpa), blackcurrant (e.g. from the species Ribes nigrum), chamomile (e.g. from the species Matricaria chamomilla and/or Matricara recutita), rosemary (e.g. from the species Rosmarinus officinalis), sage (e.g. from the species Salvia officinalis), pomegranate (e.g. from the species Punica granatum), and combinations thereof. Alternate species of the exemplified genera above may also be suitable. Other optional extracts and additives suitable for the nutritional supplement are described in U.S. Pat. Nos. 6,413,545; 6,967,206; 6,989,161; 7,348,034; 7,588,785; and 8,623,335; and U.S. Published Patent Applications 2004/0097432; 2007/0036742; 2013/0196009; 2015/0086686; and 2015/0313835; each of which is hereby incorporated by reference for this purpose.

Optionally, the nutritional supplement may include a pharmaceutically acceptable excipient including, but not limited to, croscarmellose sodium, maltodextrin, silicified microcrystalline cellulose, silicon dioxide, stearic acid, hydroxyl propyl methyl cellulose (HPMC), lactose, glucose, sucrose, corn starch, potato starch, cellulose acetate, ethyl cellulose and the like. Diluents and other additives such as one or more pharmaceutically acceptable adjuvants, binding agents (or binders), fillers, supports, thickening agents, taste-improving agents, coloring agents (e.g. natural colorants), preservatives, stabilizers, sweeteners, flavoring agents, regulators, emulsifiers or mixtures thereof, also may be used depending on the form of the nutritional supplement. Other conventional additives also may be utilized in the nutritional supplement including those described in the incorporated references above.

The nutritional supplement can be prepared using various methods understood in the art. In one example of preparing the nutritional supplement, the preparation method comprises the step of combining components (A), (B) and (C), optionally along with (D) one or more secondary components as described herein, to obtain the nutritional supplement. The components can be combined in any order using conventional manufacturing methods and apparatuses, e.g. a mixer, a blender, etc. The nutritional supplement may be further processed depending on a desired form, e.g. encapsulated, compressed, etc.

The nutritional supplement can be in various forms. In various embodiments, the nutritional supplement is in the form of an oral nutritional supplement. Examples of such oral forms include pills, tablets, gel tabs, granules, powders, concentrates, solutions, suspensions, capsules, or combinations thereof. In specific embodiments, the nutritional supplement is in the form of a tablet.

In various embodiments, the nutritional supplement is in the form of an oral dosage having a weight of at least about 25, alternatively a weight of from about 25 to about 5,000, alternatively about 30 to about 4,000, alternatively about 30 to about 3,000, milligrams (mg). In specific embodiments, component (A) is present in an amount of from about 5 to about 1,000, alternatively about 10 to about 1,000, mg. In further embodiments, component (B) is present in an amount of from about 5 to about 1,000, alternatively about 10 to about 1,000, mg. In further embodiments, component (C) is present in an amount of from about 5 to about 1,000, alternatively about 100 to about 1,000, mg. In a specific embodiment, each of components (A) and (B) are present in an amount of about 11 mg and component (C) is present in an amount of about 110 mg. Various subranges and amounts of each of components (A) to (C) between about 5 and about 1,000 mg, as well as amounts that are less than or greater than these amounts, are also contemplated.

The current embodiments also provide a method of activating a subject's antioxidant system. The method comprises the step of orally administering a nutritional supplement to the subject. The nutritional supplement is as disclosed herein. The subject is typically mammalian, more typically a human, and can include males and females of various ages. Typically, the subject is a teen or adult. In various embodiments, the nutritional supplement is orally administered to the subject on a periodic basis as part of a nutritional supplement regime. In further embodiments, the nutritional supplement is orally administered to the subject at least once per day, alternatively at least twice per day, according to the nutritional supplement regime.

The nutritional supplement may be administered as needed, daily, several times per day or in any suitable regimen such that the desired outcome is achieved. In the method, the frequency of administration can depend on several factors, including the desired level of antioxidant effect. Generally, a regimen includes administration of the nutritional supplement once or twice daily to include an administration in the morning and/or an administration in the evening. The amount of nutritional supplement ingested during each administration may depend on several factors including level of desired results and the specific composition.

In one embodiment, the nutritional supplement is administered orally in the form of a drink. When the nutritional supplement is orally administered in the form of a liquid, the liquid may be water-based, milk-based, tea-based, fruit juice-based, or some combination thereof. Solid and liquid formulations for internal administration according to the present invention can further comprise thickeners, including xanthum gum, carbosymethyl-cellulose, carboxyethylcellulose, hydroxyporpolcellulose, methylcellulose, microcrystalline cellulose, starches, dextrins, fermented whey, tofu, maltodextrins, polyols, including sugar alcohols (e.g. sorbitol and mannitol), carbohydrates (e.g. lactose), propylene glycol alginate, gellan gum, guar, pectin, tragacanth gum, gum acacia, locust bean gum, gum arabic, gelatin, as well as mixtures of these thickeners.

Solid and liquid (e.g. food and beverage) formulations including the nutritional supplement may contain an effective amount of one or more sweeteners, including carbohydrate sweeteners and natural and/or artificial no/low calorie sweeteners. The amount of the sweetener used in the formulations of the current embodiments will vary, but typically depends on the type of sweetener used and the sweetness intensity desired.

The following additional embodiments are provided, the numbering of which is not to be construed as designating levels of importance.

Embodiment 1 relates to a nutritional supplement comprising: (A) glabridin; (B) licochalcone A; and (C) licorice root flavonoids; wherein components (A), (B) and (C) are present in a combined amount sufficient to activate a subject's antioxidant system after ingestion of the nutritional supplement; and wherein components (A), (B) and (C) are present in a weight ratio (A:B:C) such that their combined effect is greater than the sum of their separate effects with respect to activation of the subject's antioxidant system after ingestion of the nutritional supplement.

Embodiment 2 relates to a nutritional supplement comprising: (A) a first licorice root extract comprising glabridin; (B) a second licorice root extract different from the first licorice root extract, the second licorice root extract comprising licochalcone A; and (C) a third licorice root extract different from the first licorice root extract and different from the second licorice root extract, the third licorice root extract comprising flavonoids; wherein the first, second and third licorice root extracts are present in a combined amount sufficient to activate a subject's antioxidant system after ingestion of the nutritional supplement; and wherein the first, second and third licorice root extracts are present in a weight ratio such that their combined effect is greater than the sum of their separate effects with respect to activation of the subject's antioxidant system after ingestion of the nutritional supplement.

Embodiment 3 relates to Embodiment 1 or 2, wherein component (A) is obtained from the species Glycyrrhiza glabra L.

Embodiment 4 relates to any one of the preceding Embodiments, wherein component (B) is obtained from the species Glycyrrhiza inflate B.

Embodiment 5 relates to any one of the preceding Embodiments, wherein component (C) is obtained from the species Glycyrrhiza inflate B.

Embodiment 6 relates to any one of the preceding Embodiments, wherein component (A) is present in an amount of from about 5 to about 45 parts by weight, alternatively about 8 to about 42 parts by weight, based on 100 parts by weight of the nutritional supplement.

Embodiment 7 relates to any one of the preceding Embodiments, wherein component (B) is present in an amount of from about 5 to about 45 parts by weight, alternatively about 8 to about 42 parts by weight, based on 100 parts by weight of the nutritional supplement.

Embodiment 8 relates to any one of the preceding Embodiments, wherein component (C) is present in an amount of from about 10 to about 90 parts by weight, alternatively about 16 to about 83 parts by weight, based on 100 parts by weight of the nutritional supplement.

Embodiment 9 relates to any one of the preceding Embodiments, wherein component (C) comprises at least one of glabridin, licochalcone A, glycyrrhizin and liquiritin, alternatively component (C) comprises glabridin, licochalcone A, glycyrrhizin, and liquiritin.

Embodiment 10 relates to any one of the preceding Embodiments, wherein component (A) is present in an amount less than a combined amount of components (B) and (C).

Embodiment 11 relates to any one of Embodiments 1 to 10, wherein components (A), (B) and (C) are present in a weight ratio (A:B:C) of about 1:3:3, alternately about 1:2.6:2.6.

Embodiment 12 relates to any one of Embodiments 1 to 10, wherein components (A), (B) and (C) are present in a weight ratio (A:B:C) of about 3:3:1, alternatively about 2.6:2.6:1.

Embodiment 13 relates to any one of Embodiments 1 to 10, wherein components (A), (B) and (C) are present in a weight ratio (A:B:C) of about 1:1:10, alternatively about 1:1:10.4.

Embodiment 14 relates to any one of Embodiments, wherein the components (A), (B) and (C) are present in a weight ratio (A:B:C) of about 3:1:3, alternatively about 2.6:1:2.6.

Embodiment 15 relates to any one of Embodiments 1 to 10, wherein components (A), (B) and (C) are present in a weight ratio (A:B:C) of about 1:1:5.

Embodiment 16 relates to any one of Embodiments 1 to 10, wherein components (A), (B) and (C) are present in a weight ratio (A:B:C) of about 1:1:1.

Embodiment 17 relates to any one of the preceding Embodiments, wherein components (A), (B) and (C) are each individually derived via extraction of root material obtained from different licorice plants, alternatively via ethanolic extraction of dry root material obtained from different licorice plants.

Embodiment 18 relates to Embodiment 17, wherein component (B) is obtained via a first extraction method and component (C) is obtained via a second extraction method different from the first extraction method, and wherein relative to the second extraction method, the first extraction method includes an additional step of purifying the licorice extract via a column.

Embodiment 19 relates to any one of the preceding Embodiments, consisting essentially of components (A), (B) and (C), alternatively consisting of components (A), (B) and (C).

Embodiment 20 relates to any one of the preceding Embodiments, further comprising (D) at least one additive, alternatively at least one of an adjuvant, a binder, an excipient and other extract, each being different from components (A), (B) and (C).

Embodiment 21 relates to any one of the preceding Embodiments, in the form of an oral dosage having a weight of at least about 25, alternatively a weight of from about 25 to about 5,000, milligrams.

Embodiment 22 relates to any one of the preceding Embodiments, in the form of an oral nutritional supplement, alternatively in the form of an oral pill, tablet, gel tab, granule, powder, concentrate, solution, suspension, capsule, or combinations thereof.

Embodiment 23 relates to any one of the preceding Embodiments, wherein after ingestion the subject's antioxidant response element (ARE) activates.

Embodiment 24 relates to any one of the preceding Embodiments, wherein the subject is mammalian, alternatively human.

Embodiment 25 relates to a method of activating a subject's antioxidant system, the method comprising the step of: orally administering a nutritional supplement to the subject; wherein the nutritional supplement is as set forth in any one of Embodiments 1 to 24.

Embodiment 26 relates to Embodiment 25, wherein the nutritional supplement is orally administered to the subject on a periodic basis as part of a nutritional supplement regime.

Embodiment 27 relates to Embodiment 26, wherein the nutritional supplement is orally administered to the subject at least once per day, alternatively at least twice per day, according to the nutritional supplement regime.

The individual components of these embodiments are generally as described above. All combinations of the aforementioned embodiments are hereby expressly contemplated.

The following examples, illustrating the nutritional supplement and components thereof, are intended to illustrate and not to limit the invention.

### EXAMPLES

Three different licorice root extracts are obtained from BGG. The first licorice root extract is derived from ethanolic extraction of root material from the species Glycyrrhiza glabra L. and is designated as “Glabridin 4% (GN-04)” or simply “G” in certain Figures and/or below. The first licorice root extract is generally standardized at about 4% glabridin. The second licorice root extract is derived from ethanolic extraction of root material from the species Glycyrrhiza inflata B. and is designated as “Licochalcone A 21% (GA-105)” or simply “L” in certain Figures and/or below. The second licorice root extract is generally standardized at about 21% licochalcone A. The third licorice root extract is derived from ethanolic extraction of root material from the species Glycyrrhiza inflate B. and is designated as “Licorice flavonoids (GD-053)” or simply “F” in certain Figures and/or below.

Each of licorice root extracts is analyzed via HPLC via methodology understood in the art. FIG. 1 is a chromatogram for Glabridin4% (GN-04). FIG. 2 is a chromatogram for Licochalcone A 21% (GA-105), and FIG. 3 is a chromatogram for Licorice flavonoids (GD-053). Even with use of different HPLC methods, it can be appreciated that the licorice root extracts are still distinguishable from each other based on different chromatograms (see, for example, FIGS. 1A, 2A and 3A relative to 1B, 2B and 3B). The present invention is not limited to a particular HPLC method or technique.

The following peaks are observed in FIGS. 1A, 2A and 3A:

TABLE 1

| Peak number | Constituent |
|---|---|
| 1 | Liquiritin |
| 2 | Glycyrrhizic acid |
| 3 | Licochalcone A |
| 4 | Glabridin |

An assay was created by transfecting human hepatocytes with a vector containing four repeats of the ARE DNA sequence (5′-GTGACTCAGCA-3′) upstream of a minimal promoter, and firefly luciferase expression as the reporter. This cell line was treated with various licorice root extracts, individually and in combination (or blends), for 48 hours.

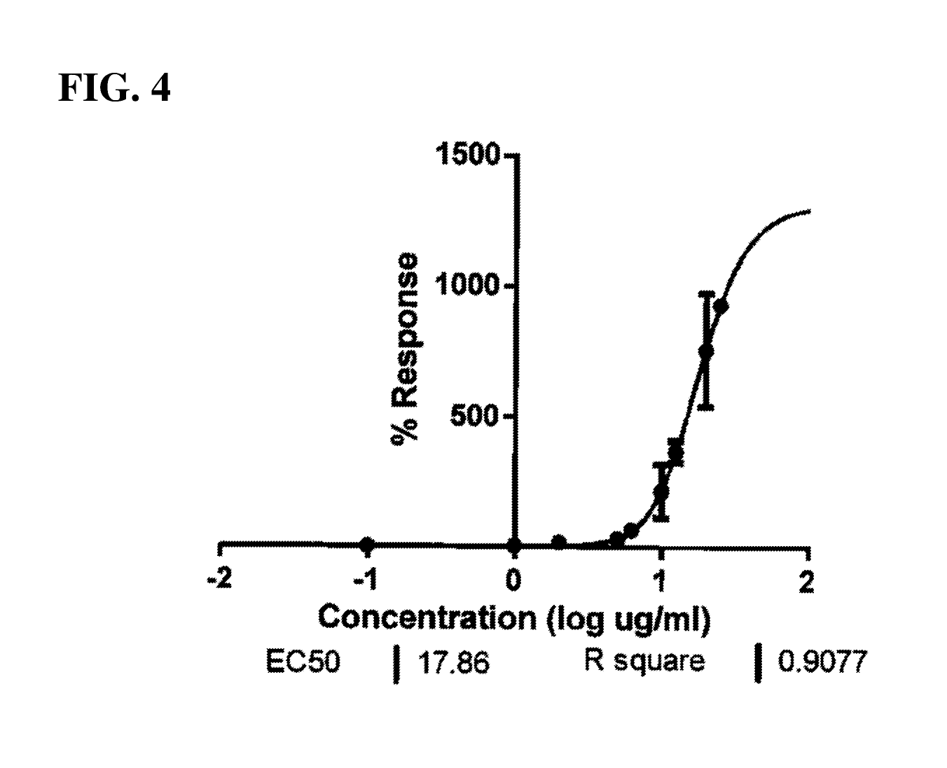

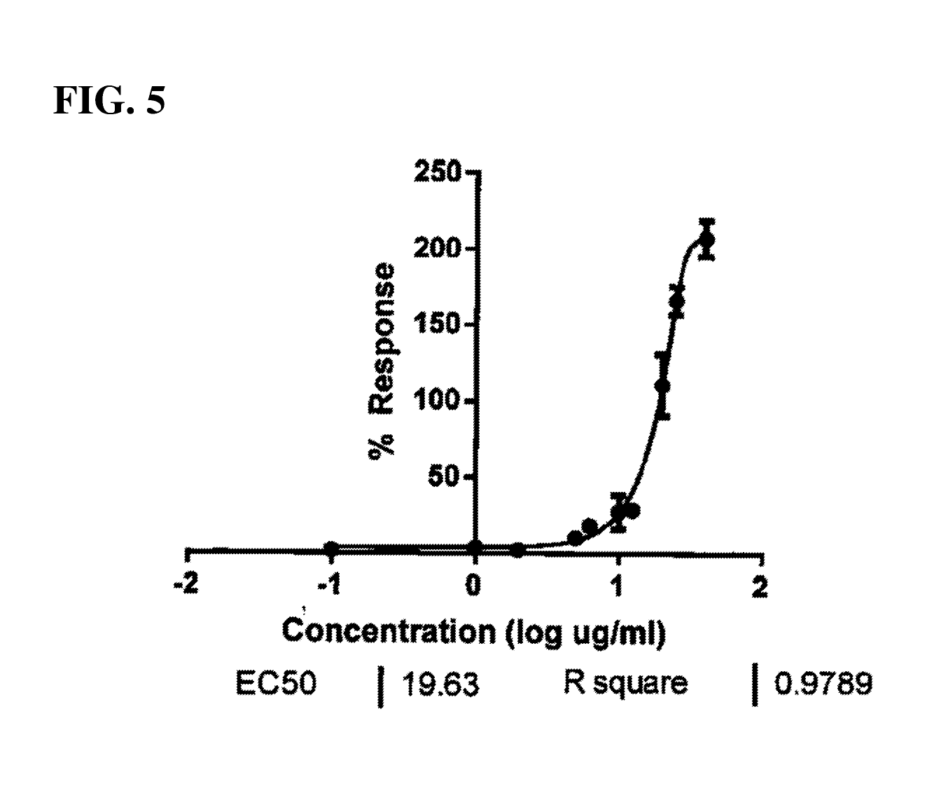

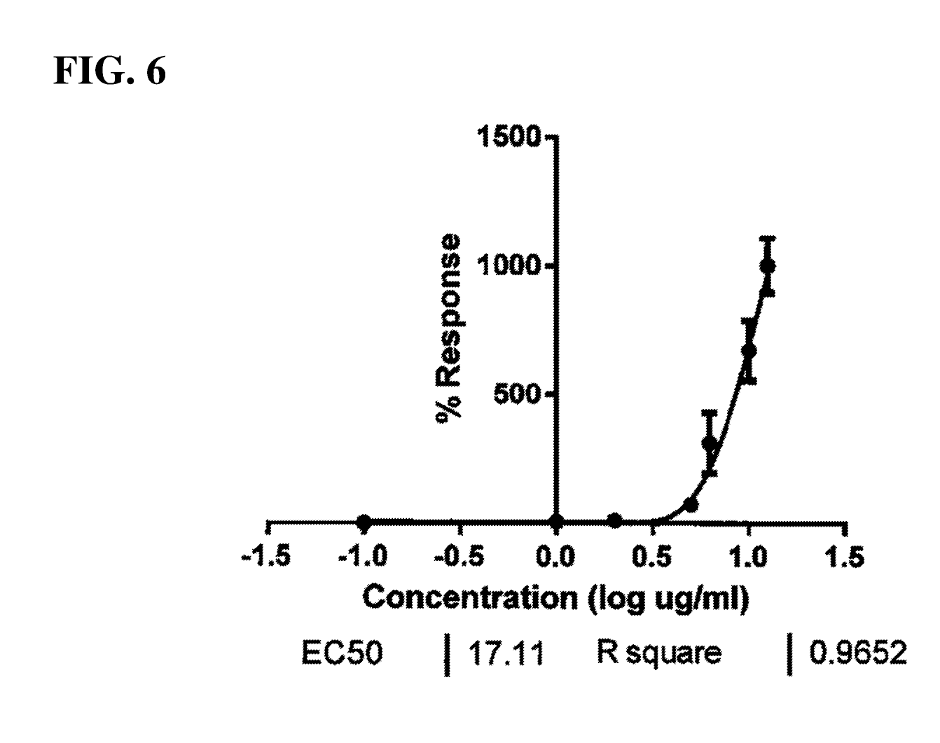

Upon lysis and addition of luciferase substrate, luminescence values were measured and the percent response was normalized to the known ARE activator, sulforaphane. Initial testing indicated each licorice root extract had a stimulatory effect on ARE, ranging from approximately 200-1000% maximal response as illustrated in FIGS. 4 to 6. FIG. 4 is a dose response curve of Licorice flavonoids (GD-053). FIG. 5 is a dose response curve of Glabridin 4% (GN-04). FIG. 6 is a dose response curve of Licochalcone A 21% (GA-105).

As each individual licorice root extract possesses an Effective Concentration value (EC50) of less than 25 μg/ml, the licorice root extracts were tested in various combinations to look for possible synergistic effects on ARE activation.

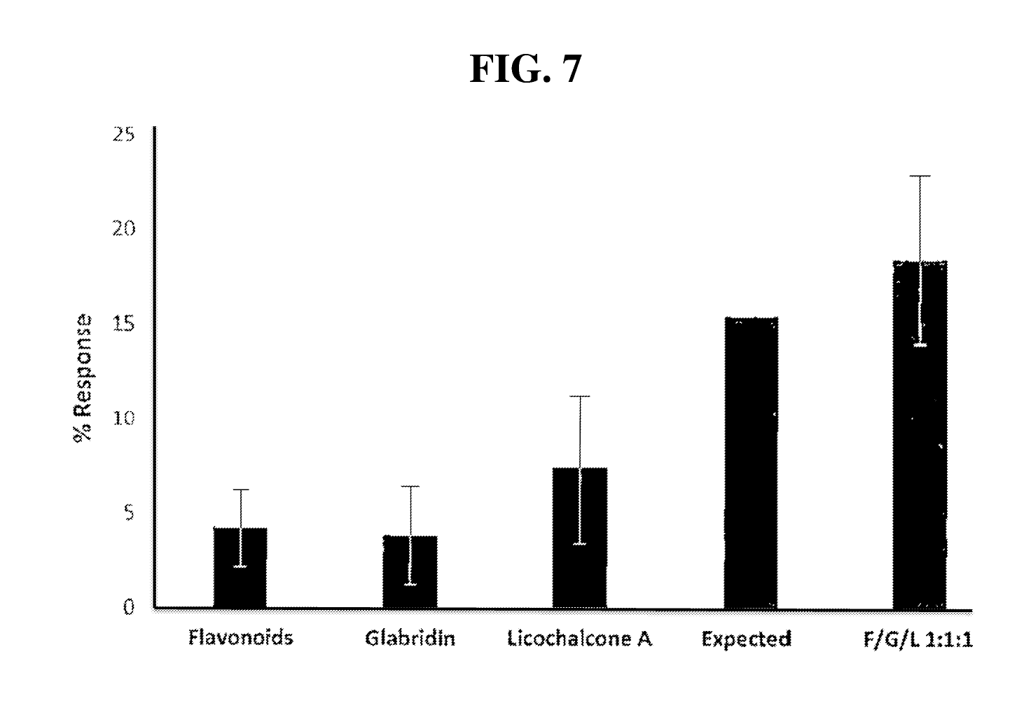

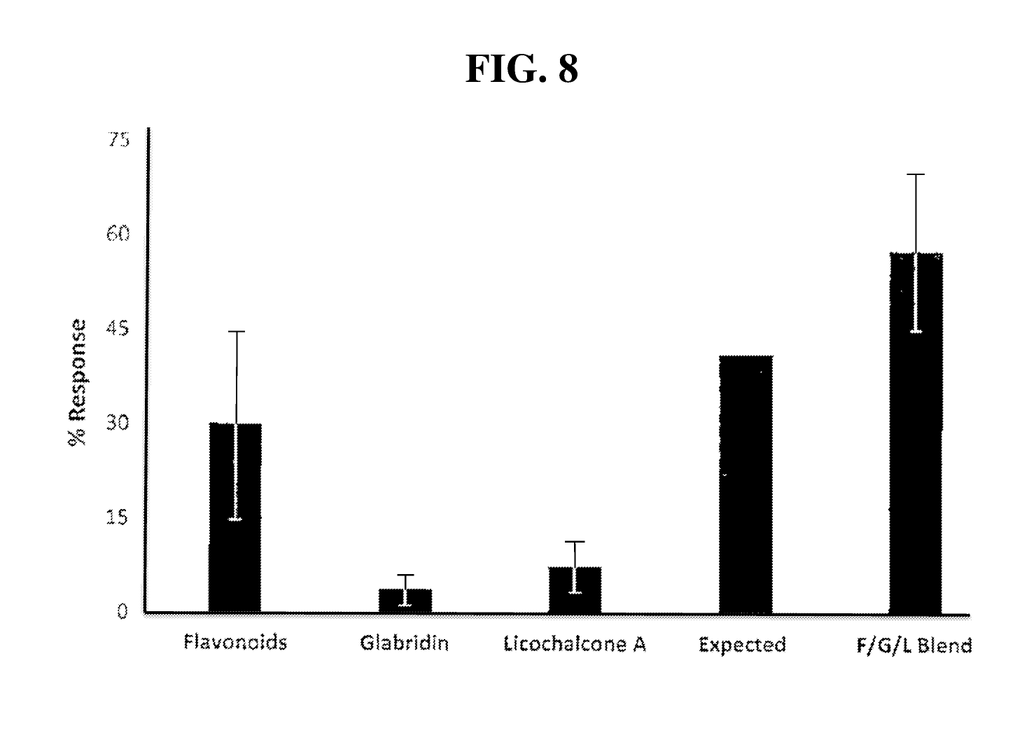

Referring to FIG. 7, at a 1:1:1 weight ratio (e.g. ˜33 wt % each), the licorice root extract combination yields a 119% response above the expected additive effect of 15.45%. Referring to FIG. 8, when the licorice root extract combination includes 71 wt % total flavonoids and equal parts glabridin and licochalcone A (14 wt % each), the combined response increases slightly to 140% of the expected additive effect.

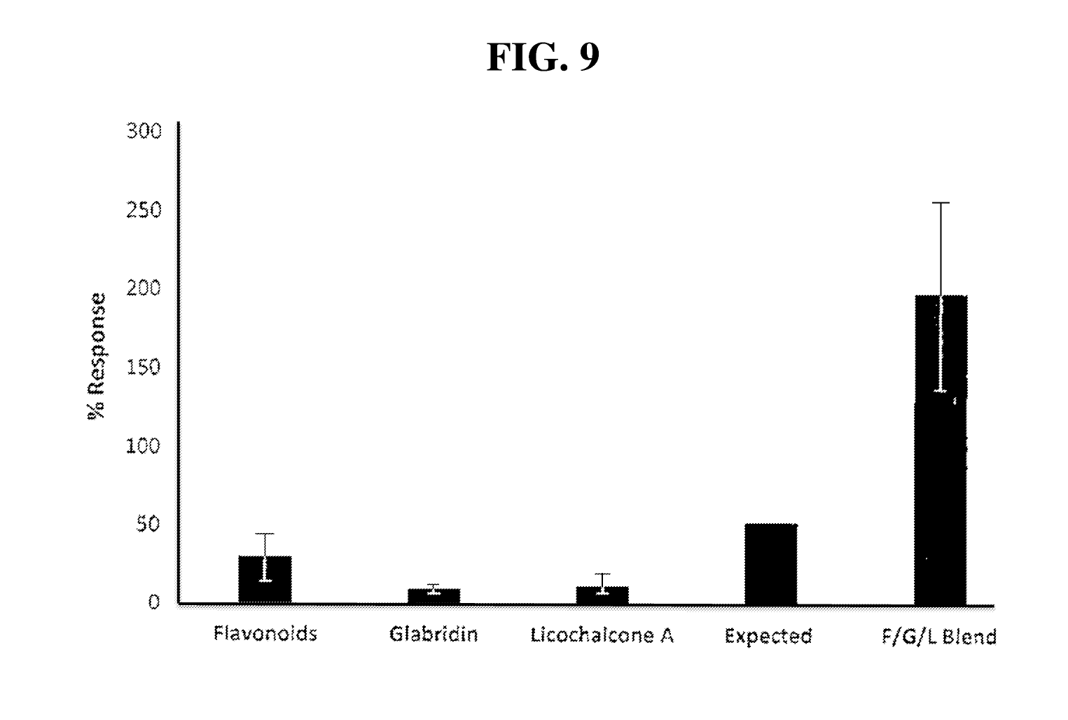

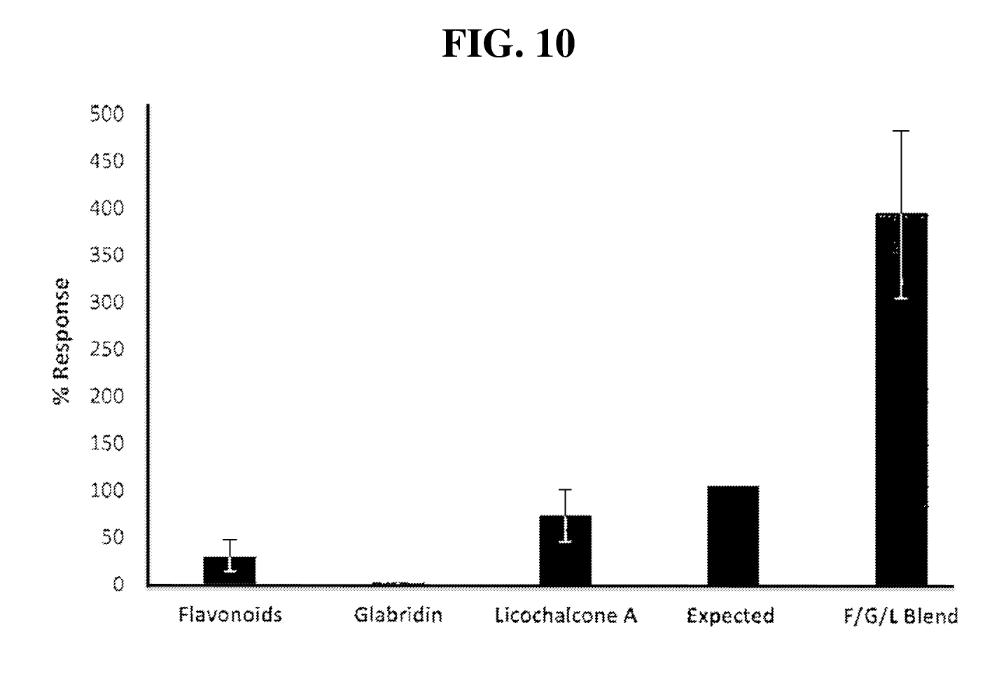

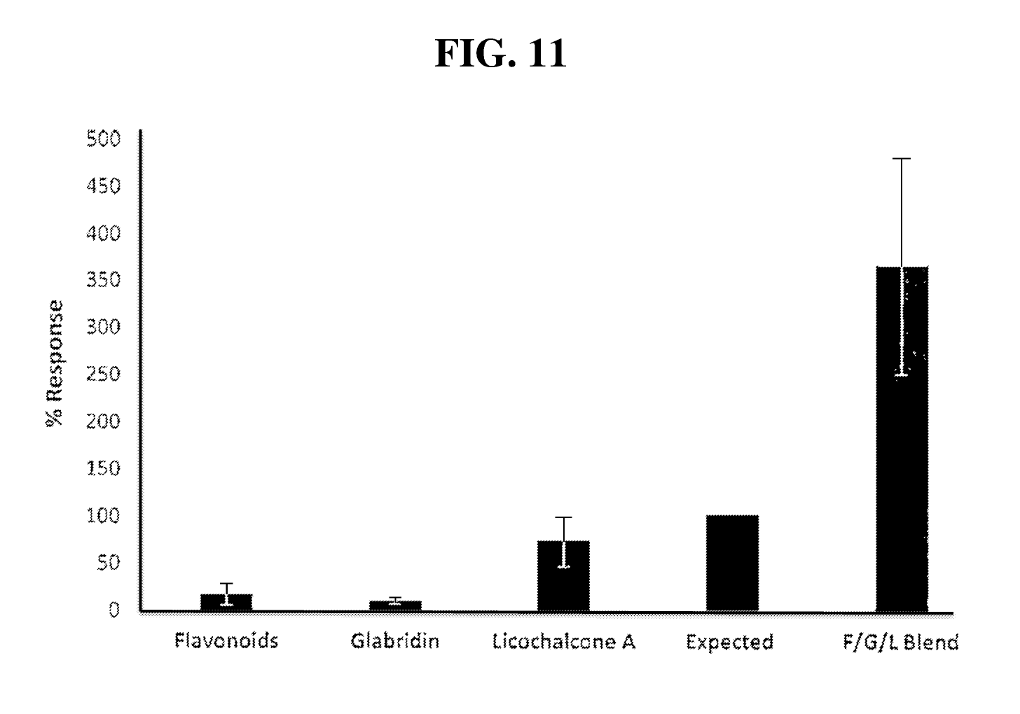

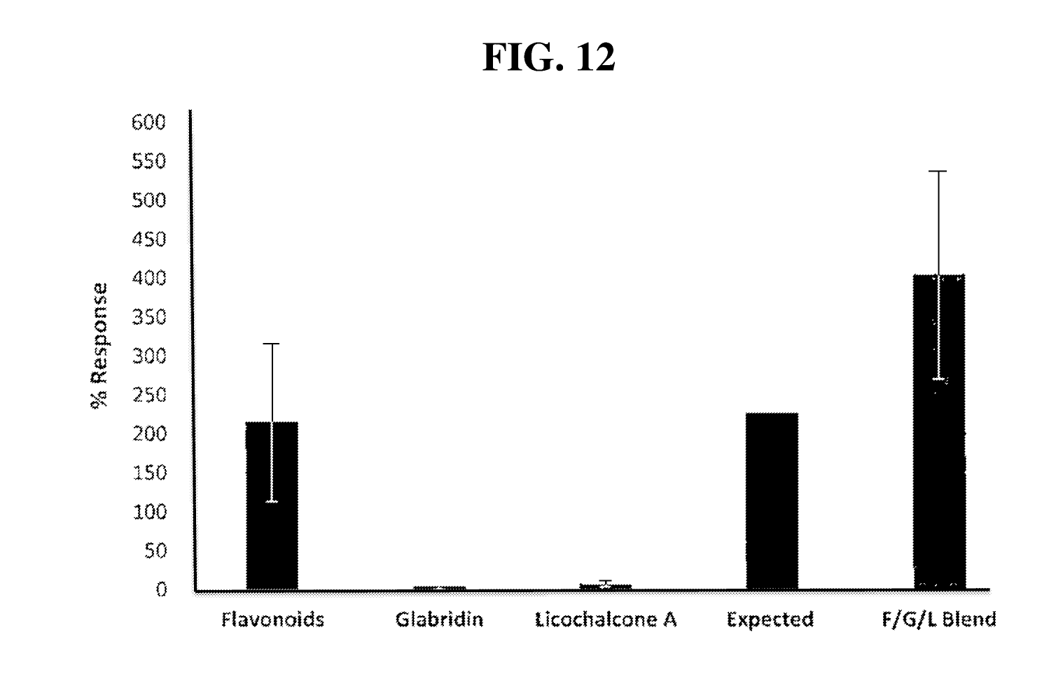

Referring to FIG. 9, synergy is also observed when flavonoids and glabridin are each present in the composition at 42 wt % and licochalcone A is present at 16 wt %. Referring to FIG. 10, synergy is also observed when flavonoids and licochalcone A are each present in the composition at 42 wt % and glabridin is present at 16 wt %. Referring to FIG. 11, synergy is also observed when glabridin and licochalcone A are each present in the composition at 42 wt % and flavonoids is present at 16 wt %. Referring to FIG. 12, synergy is also observed when glabridin and licochalcone A are each present in the composition at 8 wt % and flavonoids is present at 83 wt %.

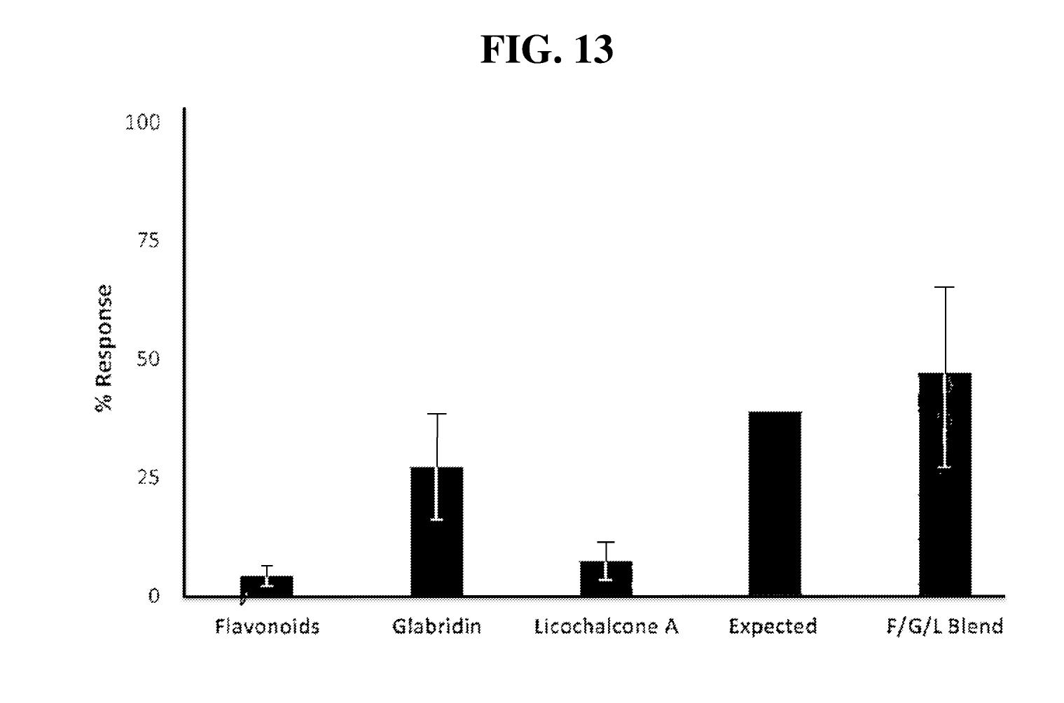

Referring to FIG. 13, synergy is not observed when flavonoids and licochalcone A are each present in the composition at 8 wt % and glabridin is present at 83 wt %. Specifically, when glabridin comprises a majority of the licorice blend and flavonoids and licochalcone A are minimal in the composition, there is a lack of synergistic activation of the ARE and the overall response diminishes below 100% compared to the control.

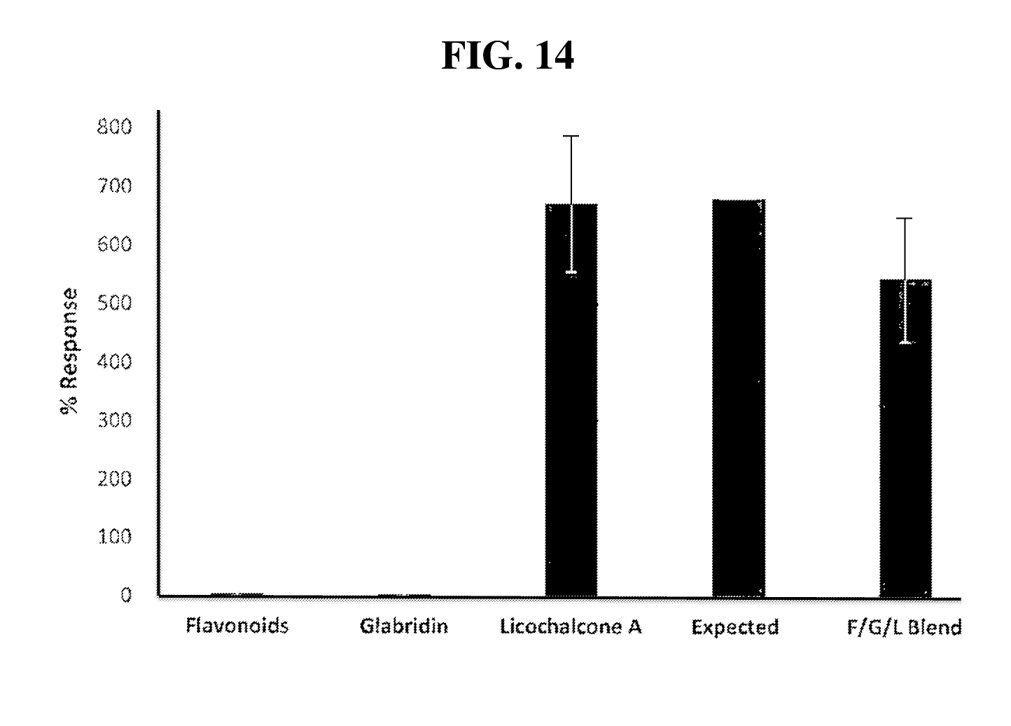

Referring to FIG. 14, synergy is not observed when flavonoids and glabridin are each present in the composition at 8 wt % and licochalcone A is present at 83 wt %. Specifically, the expected additive response is 680%, but when combined the licorice root extract blend yields only a 545% response, a non-synergistic response. Even though licochalcone A boosts ARE activity, all three licorice root extracts are required in specific concentrations to achieve synergy.

As illustrated above along with the corresponding Figures, the three licorice root extracts produce a synergistic activation of the ARE when used in combination with at least the general ranges of about 16-83 wt % for licorice flavonoids, about 8-42 wt % for glabridin and about 8-42 wt % for licochalcone A.

The terms “comprising” or “comprise” are used herein in their broadest sense to mean and encompass the notions of “including,” “include,” “consist(ing) essentially of,” and “consist(ing) of”. The use of “for example,” “e.g.,” “such as,” and “including” to list illustrative examples does not limit to only the listed examples. Thus, “for example” or “such as” means “for example, but not limited to” or “such as, but not limited to” and encompasses other similar or equivalent examples. The term “about” as used herein serves to reasonably encompass or describe minor variations in numerical values measured by instrumental analysis or as a result of sample handling. Such minor variations may be in the order of ±0-10, ±0-5, or ±0-2.5, % of the numerical values. Further, The term “about” applies to both numerical values when associated with a range of values. Moreover, the term “about” may apply to numerical values even when not explicitly stated.

Generally, as used herein a hyphen “-” or dash “-” in a range of values is “to” or “through”; a “>” is “above” or “greater-than”; a “≧” is “at least” or “greater-than or equal to”; a “<” is “below” or “less-than”; and a “≦” is “at most” or “less-than or equal to.” On an individual basis, each of the aforementioned applications for patent, patents, and/or patent application publications, is expressly incorporated herein by reference in its entirety in one or more non-limiting embodiments.

It is to be understood that the appended claims are not limited to express and particular compounds, compositions, or methods described in the detailed description, which may vary between particular embodiments which fall within the scope of the appended claims. With respect to any Markush groups relied upon herein for describing particular features or aspects of various embodiments, it is to be appreciated that different, special, and/or unexpected results may be obtained from each member of the respective Markush group independent from all other Markush members. Each member of a Markush group may be relied upon individually and or in combination and provides adequate support for specific embodiments within the scope of the appended claims.

It is also to be understood that any ranges and subranges relied upon in describing various embodiments of the present invention independently and collectively fall within the scope of the appended claims, and are understood to describe and contemplate all ranges including whole and/or fractional values therein, even if such values are not expressly written herein. One of skill in the art readily recognizes that the enumerated ranges and subranges sufficiently describe and enable various embodiments of the present invention, and such ranges and subranges may be further delineated into relevant halves, thirds, quarters, fifths, and so on. As just one example, a range “of from 0.1 to 0.9” may be further delineated into a lower third, i.e., from 0.1 to 0.3, a middle third, i.e., from 0.4 to 0.6, and an upper third, i.e., from 0.7 to 0.9, which individually and collectively are within the scope of the appended claims, and may be relied upon individually and/or collectively and provide adequate support for specific embodiments within the scope of the appended claims. In addition, with respect to the language which defines or modifies a range, such as “at least,” “greater than,” “less than,” “no more than,” and the like, it is to be understood that such language includes subranges and/or an upper or lower limit. As another example, a range of “at least 10” inherently includes a subrange of from at least 10 to 35, a subrange of from at least 10 to 25, a subrange of from 25 to 35, and so on, and each subrange may be relied upon individually and/or collectively and provides adequate support for specific embodiments within the scope of the appended claims. Finally, an individual number within a disclosed range may be relied upon and provides adequate support for specific embodiments within the scope of the appended claims. For example, a range “of from 1 to 9” includes various individual integers, such as 3, as well as individual numbers including a decimal point (or fraction), such as 4.1, which may be relied upon and provide adequate support for specific embodiments within the scope of the appended claims.

The present embodiments have been described herein in an illustrative manner, and it is to be understood that the terminology which has been used is intended to be in the nature of words of description rather than of limitation. Many modifications and variations of the present invention are possible in light of the above teachings. The present embodiments may be practiced otherwise than as specifically described within the scope of the appended claims. The subject matter of all combinations of independent and dependent claims, both single and multiple dependent, is herein expressly contemplated.

What is claimed is:

1. A nutritional supplement comprising:
(A) glabridin;
(B) licochalcone A; and
(C) licorice root flavonoids;
wherein components (A), (B) and (C) are present in a combined amount sufficient to activate a subject's antioxidant system after ingestion of said nutritional supplement; and
wherein components (A), (B) and (C) are present in a weight ratio (A:B:C) such that their combined effect is greater than the sum of their separate effects with respect to activation of the subject's antioxidant system after ingestion of said nutritional supplement.

2. The nutritional supplement of claim 1,
wherein component (A) is obtained from the species Glycyrrhiza glabra L.

3. The nutritional supplement of claim 2,
wherein component (B) is obtained from the species Glycyrrhiza inflate B.

4. The nutritional supplement of claim 3,
wherein component (C) is obtained from the species Glycyrrhiza inflate B.

5. The nutritional supplement of claim 1,
wherein component (A) is present in an amount of from about 5 to about 45 parts by weight, alternatively about 8 to about 2 parts by weight, based on 100 parts by weight of said nutritional supplement.

6. The nutritional supplement of claim 1,
wherein component (B) is present in an amount of from about 5 to about 45 parts by weight, alternatively about 8 to about 42 parts by weight, based on 100 parts by weight of said nutritional supplement.

7. The nutritional supplement of claim 1,
wherein component (C) is present in an amount of from about 10 to about 90 parts by weight, alternatively about 16 to about 83 parts by weight, based on 100 parts by weight of said nutritional supplement.

8. The nutritional supplement of claim 1,
wherein component (A) is present in an amount less than a combined amount of components (B) and (C).

9. The nutritional supplement of claim 1,
wherein components (A), (B) and (C) are present in a weight ratio (A:B:C) of about 1:2.6:2.6.

10. The nutritional supplement of claim 1,
wherein components (A), (B) and (C) are present in a weight ratio (A:B:C) of about 2.6:2.6:1.

11. The nutritional supplement of claim 1,
wherein components (A), (B) and (C) are present in a weight ratio (A:B:C) of about 1:1:10.

12. The nutritional supplement of claim 1,
wherein said components (A), (B) and (C) are present in a weight ratio (A:B:C) of about 3:1:3.

13. The nutritional supplement of claim 1, wherein said components (A), (B) and (C) are present in a weight ratio (A:B:C) of about 1:1:5.

14. The nutritional supplement of claim 1,
wherein after ingestion the subject's antioxidant response element (ARE) activates.

15. A method of activating a subject's antioxidant system, said method comprising the step of: orally administering the nutritional supplement of claim 1 to a subject.

16. The method as set forth in claim 15,
wherein the nutritional supplement is orally administered to the subject on a periodic basis as part of a nutritional supplement regime.

17. A nutritional supplement comprising:
(A) a first licorice root extract comprising glabridin;
(B) a second licorice root extract different from said first licorice root extract, said second licorice root extract comprising licochalcone A; and
(C) a third licorice root extract different from said first licorice root extract and different from said second licorice root extract, said third licorice root extract comprising flavonoids;
wherein said first, second and third licorice root extracts are present in a combined amount sufficient to activate a subject's antioxidant system after ingestion of said nutritional supplement; and
wherein said first, second and third licorice root extracts are present in a weight ratio such that their combined effect is greater than the sum of their separate effects with respect to activation of the subject's antioxidant system after ingestion of said nutritional supplement.

18. The nutritional supplement of claim 17,
wherein component (A) is obtained from the species Glycyrrhiza glabra L.

19. The nutritional supplement of claim 18,
wherein component (B) is obtained from the species Glycyrrhiza inflate B.

20. The nutritional supplement of claim 19,
wherein component (C) is obtained from the species Glycyrrhiza inflate B.
